(19) United States
(12) Patent Application Publication
(10) Pub. No.: US 2023/0329532 A1
(43) Pub. Date: Oct. 19, 2023

(54) Optical Phase Imaging Device Optimization Methods

(71) Applicant: Georgia Tech Research Corporation, Atlanta, GA (US)

(72) Inventors: Francisco E. Robles, Atlanta, GA (US); Zhe Guang, Atlanta, GA (US)

(21) Appl. No.: 18/300,965

(22) Filed: Apr. 14, 2023

Related U.S. Application Data
(60) Provisional application No. 63/330,899, filed on Apr. 14, 2022.

(51) Int. Cl. A61B 1/00; A61B 5/00

(52) U.S. Cl. CPC A61B 1/00163; A61B 5/0059

(57) ABSTRACT

The disclosure provides a method of determining a desired set of parameters for an imaging system to image a sample, comprising: choosing a first set of parameters for the imaging system; simulating light scattering properties of the sample when imaging the sample using the imaging system having the first set of parameters; determining a first SNR when imaging the sample using the imaging system having the first set of parameters; choosing a second set of parameters for the imaging system; simulating light scattering properties of the sample when imaging the sample using the imaging system having the second set of parameters; determining a second SNR when imaging the sample using the imaging system having the second set of parameters; determining a desired SNR, the desired SNR being the greater of the first SNR and second SNR; and selecting a desired set of parameters corresponding to the desired SNR.

### CROSS-REFERENCE TO RELATED APPLICATIONS

This application claims the benefit of U.S. Provisional Application Serial No. 63/330,899 filed on 14 Apr. 2022, which is incorporated herein by reference in its entirety as if fully set forth below.

### GOVERNMENT LICENSE RIGHTS

This invention was made with government support under NS117067, and CA223853 awarded by the National Institutes of Health, and 1752011 awarded by the National Science Foundation. The government has certain rights in the invention.

### FIELD OF THE DISCLOSURE

The various embodiments of the present disclosure relate generally to imaging systems and methods of optimizing the same.

### BACKGROUND

Quantitative phase imaging (QPI) is a wide-field, label-free imaging modality that uses differences in optical path length to quantify cellular and sub-cellular structures with nanometer scale sensitivity. Unlike other optical imaging methods used to visualize tissue structures with sub-cellar resolution (e.g., confocal microscopy, multiphoton imaging, and fluorescence microscopy), QPI does not require labels, stains, or complex systems such as high-power lasers. Further, QPI yields unique quantitative biological information related to dry mass which can be used to assess cellular/tissue structure and dynamic activity to study fundamental biological processes, as well as diseases. However, QPI utilizes a transmission-based system which sets important limitations on the thickness and transparency of the samples that can be analyzed with this method. The restriction to a transmissive geometry and thin samples has prevented the use of QPI in many medical/clinical applications, including endoscopic applications. Indeed, achieving quantitative phase contrast through a compact, flexible fiber-based system is highly desirable and could be transformative for many medical applications given QPI’s access to cellular and subcellular structures without labels or dyes.

### BRIEF SUMMARY

An exemplary embodiment of the present disclosure provides a method of determining a desired set of parameters for an imaging system to image a sample. The method can comprise: choosing a first set of parameters for the imaging system; simulating light scattering properties of the sample when imaging the sample using the imaging system having the first set of parameters; determining a first signal-to-noise ratio (SNR) when imaging the sample using the imaging system having the first set of parameters; choosing a second set of parameters for the imaging system; simulating light scattering properties of the sample when imaging the sample using the imaging system having the second set of parameters; determining a second SNR when imaging the sample using the imaging system having the second set of parameters; determining a desired SNR, the desired SNR being the greater of the first SNR and second SNR; and selecting a desired set of parameters, the desired set of parameters being one of the first set of parameters and the second set of parameters corresponding to the desired SNR.

In any of the embodiments disclosed herein, simulating light scattering properties of the sample when imaging the sample using the imaging system having the first set of parameters and simulating light scattering properties of the sample when imaging the sample using the imaging system having the second set of parameters can each comprise simulating a measurement of the number of photons collected at a detector of the imaging device when imaging the sample using the imaging system having the first and second sets of parameters, respectively.

In any of the embodiments disclosed herein, simulating light scattering properties of the sample when imaging the sample using the imaging system having the first set of parameters and simulating light scattering properties of the sample when imaging the sample using the imaging system having the second set of parameters can each comprise simulating a measurement of an oblique angle of scattered photons incident a detector of the imaging sample when imaging the sample using the imaging system having the first and second sets of parameters, respectively.

In any of the embodiments disclosed herein, determining a first SNR and determining a second SNR can each comprise calculating an optical phase transfer function for the imaging system when imaging the sample using the imaging system having the first and second sets of parameters, respectively.

In any of the embodiments disclosed herein, determining a first SNR and determining a second SNR can each further comprise calculating a slope of the optical transfer functions.

In any of the embodiments disclosed herein, determining a first SNR and determining a second SNR can each further comprise multiplying the slope of the corresponding optical transfer function by the square root of the number of photons collected at the detector of the imaging device when imaging the sample using the imaging system having the first and second sets of parameters, respectively.

In any of the embodiments disclosed herein, each of the first set of parameters and second set of parameters can comprise an illumination wavelength.

In any of the embodiments disclosed herein, each of the first set of parameters and second set of parameters can comprise a lateral separation distance.

In any of the embodiments disclosed herein, each of the first set of parameters and second set of parameters can comprise an axial separation distance between an MMF and GRIN lens.

In any of the embodiments disclosed herein, each of the first set of parameters and second set of parameters can comprise a MMF illuminating angle.

In any of the embodiments disclosed herein, each of the first set of parameters and second set of parameters can comprise a MMF NA.

In any of the embodiments disclosed herein, the imaging system can be a quantitative oblique back-illumination microscopy (qOBM) system.

In any of the embodiments disclosed herein, the imaging system can be an endoscopic oblique back illumination imaging system.

Another embodiment of the present disclosure provides a method of determining a desired set of parameters for an imaging system to image a sample. The method can comprise: simulating imaging a sample, comprising one or more iterations of: choosing a unique set of parameters for the imaging system; simulating light scattering properties of the sample when imaging the sample using the imaging system having the unique set of parameters; and determining a signal-to-noise ratio (SNR) when imaging the sample using the imaging system having the unique set of parameters; determining a desired SNR from the determined SNRs; and selecting a desired set of parameters, the desired set of parameters being the unique set of parameters corresponding to the desired SNR.

In any of the embodiments disclosed herein, simulating light scattering properties of the sample when imaging the sample using the imaging system having the unique set of parameters can comprise simulating a measurement of the number of photons collected at a detector of the imaging device when imaging the sample using the imaging system having the unique set of parameters.

In any of the embodiments disclosed herein, simulating light scattering properties of the sample when imaging the sample using the imaging system having the unique set of parameters can comprise simulating a measurement of an oblique angle of scattered photons incident a detector of the imaging sample when imaging the sample using the imaging system having the unique set of parameters.

In any of the embodiments disclosed herein, determining the SNR can comprise calculating an optical phase transfer function for the imaging system when imaging the sample using the imaging system having the unique set of parameters.

In any of the embodiments disclosed herein, determining the SNR can further comprise calculating a slope of the optical transfer function.

In any of the embodiments disclosed herein, determining the SNR can further comprise multiplying the slope of the optical transfer function by the square root of the number of photons collected at the detector of the imaging device when imaging the sample using the imaging system having the unique set of parameters.

In any of the embodiments disclosed herein, the unique set of parameters can comprise one or more selected from the following: an illumination wavelength; a lateral separation distance; an axial separation distance between an MMF and GRIN lens; a MMF illuminating angle; and a MMF NA.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1A provides an experimental setup and imaging mechanism of a fiber-based qOBM system. FIG. 1B illustrates epi-illumination, used for qOBM, where multiply scattered photons within a thick sample (brain) form a banana-shaped path from the source (MMF) to the detector (GRIN lens) and produce a virtual, oblique light source at the focal plane, in which D_{L} and D_{A} are the lateral and axial separations, respectively, between MMF and GRIN lens, and θ is the fiber tilting illumination angle. The bottom-right inset of FIG. 1B shows a simulated photon illumination in the spatial-frequency (kₓ, k_{y}) domain. FIG. 1C shows 2D optical phase transfer function of the qOBM system, in which the inset shows the profile along the central dashed line, where the slope, m, near the center of the transfer function, scales with the phase sensitivity of a particular probe design.

FIG. 2A provides a flow chart to process four raw intensity images, into two DPC images, and finally into one qOBM image representing the quantitative phase. FIG. 2B provides raw intensity of 10 µm beads in water atop a glass slide, under one LED source illumination (from the left), in which beads were illuminated through oblique back-scattered light from a piece of paper under the sample. FIG. 2C provides a low-pass filtered intensity image. FIG. 2D provides a DPC image obtained from opposite horizontal illuminations. FIG. 2E provides a qOBM image showing quantitative phase. The bottom-right insets of FIGS. 2BE show zoomed-in areas, marked by the dashed squares in the main plots.

FIGS. 3A-B illustrate wavelength-dependent optimization of SNR in probe design by Monte Carlo simulations, in accordance with some embodiments of the present disclosure. FIG. 3A illustrates SNR vs. illumination wavelength (simulated in white matter), in which the upper-left inset plots logarithm of photon-counts (N) vs. wavelength, and the bottom-right inset plots central slope m of optical phase transfer function vs. wavelength. FIG. 3B illustrates system SNR obtained by multiplying SNR in FIG. 3A with the square root of QE of the camera, in which the bottom inset plots QE vs. wavelength (data from vendor).

FIGS. 4A-D provide plots SNRs simulated over the lateral separation distance D_{L} and axial separation distance D_{A} between GRIN lens and MMF, in accordance with some embodiments of the present disclosure. SNRs plots are shown for (FIG. 4A) white matter, (FIG. 4B) grey matter, (FIG. 4C) epidermis, and (FIG. 4D) breast tissue. The insets show photon-count log(N) and central slope m variation over D_{L} and D_{A}. Circled numbers indicate three different geometries investigated experimentally. All SNR values are normalized to geometry^{②} value in grey matter.

FIGS. 5A-F provide plots of SNRs simulated over initial fiber illumination angle or fiber NA, and over lateral separation, in accordance with some embodiments of the present disclosure. FIGS. 5A, 5C, & 5E plot SNR as a function of lateral separation D_{L} and illumination angle, at axial separation D_{A}= 0 mm, 4 mm, 6 mm, respectively, and NA = 0.5. FIGS. 5B, 5D, & 5F plot SNR as a function of lateral separation D_{L} and fiber NA, at axial separation D_{A}= 0 mm, 4 mm, 6 mm, respectively, and illumination angle of zero degrees. In all cases, the circled numbers indicate selected geometries as in FIGS. 4A-D. All SNR values are normalized to geometry^{②} value in grey matter.

FIGS. 6A-B illustrate SNR experimental validation of Monte Carlo simulations by measuring 10 µm beads in white and grey matter scattering phantoms, in accordance with some embodiments of the present disclosure. In FIG. 6A, experiment SNRs for 3 distinct geometries (defined in FIG. 4) are drawn with solid bars and error bars, and simulations are drawn with dashed bars. In FIG. 6B, experiment SNRs as a function of illumination angle and axial separation distance D_{A} are given in solid lines, and simulated results are in dashed lines, for the grey matter phantom. The lateral separation distance D_{L} is fixed to 2.5 mm. Experiment SNR in all cases is calculated by dividing the average measured phase of 10 beads by the phase standard deviation of 10 featureless areas. All SNR values are normalized to geometry^{②} value in the grey matter phantom.

FIGS. 7A-F provide characterizations of phase sensitivity, in which a photolithographic quartz phase target with letters of 300 nm, 200 nm, and 100 nm in height is measured. FIG. 7A provides phase measurement of the target. FIG. 7B provides phase measurement of a blank area. FIG. 7C provides phase retrieved after background subtraction. FIG. 7D provides phase mapped to quartz height in air. FIG. 7E provides measured phase standard deviation over the number of averaged frames, N_{f}. The dotted lower curve follows a

$$∝ 1 / \sqrt{{N}_{f}}$$

dependence, corresponding to a Poisson noise distribution, while the dashed curve (fitted curve) follows arelation:

$$15.03 n m / \sqrt{{N}_{f}} + 3.05 \text{nm} .$$

The shaded area indicates the measured data ± standard deviation of the four featureless areas. FIG. 7F provides measured single-pixel phase fluctuation over time (3 minutes). Measured phases are from 4 selected pixels in the phase target measurement, as shown in the upper-left inset.

FIGS. 8A-H illustrate measurements of formalin-fixed rat brains from a 9L gliosarcoma tumor model. FIGS. 8A-D illustrate healthy cortex area (choroid plexus). FIGS. 8E-H illustrate dense tumor cellular area. FIGS. 8A & 8E provide low-pass filtered single-frame intensity images. FIGS. 8B & 8F provide DPC images under horizontal illumination. FIGS. 8C & 8G provide retrieved quantitative phase (qOBM images) without frame averaging. FIGS. 8D & 8H provide retrieved quantitative phase images by averaging 40 frames.

FIGS. 9A-I provide images of unstained, freshly excised, thick human brain tumor samples using the fiber-based qOBM system (FIGS. 9A, 9D, & 9G), in comparison to free-space qOBM images (FIGS. 9B, 9E, & 9H) and H&E stained slices (FIGS. 9C, 9F, & 9I; after fixation and processing. FIGS. 9A-C provide images of a capillary blood vessel with single blood cells inside, and tumor cells around. FIGS. 9D-F provide images of a large blood vessel, where blood cells are closely packed, and astrocytoma tumor cells are nearby. FIGS. 9G-I provide images of a densely packed tumor area, where cell nucleus and myelin sheath of neurons (with higher phase contrast) are visible.

FIG. 10 provides a flow chart for a method of determining a desired set of parameters for an imaging system to image a sample, in accordance with an exemplary embodiment of the present disclosure.

FIG. 11 provides a flow chart for a method of determining a desired set of parameters for an imaging system to image a sample, in accordance with an exemplary embodiment of the present disclosure.

FIG. 12 provides a block diagram of an exemplary computing device that can be used in accordance with various embodiments of the present disclosure.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

To overcome the limitations of QPI discussed above, the inventors recently introduced a technique called quantitative oblique back-illumination microscopy (qOBM), which yields tomographic quantitative phase information of thick scattering samples with epi-illumination. This technique is disclosed in U.S. Pat. App. Publication No. 2021/0025818, entitled “Cell Imaging Systems and Methods,” which is incorporated herein by reference in its entirety as if fully set forth below.

But there is still a need for a method of determining an optimized set of parameters of the imaging device for a particular sample to be images. Accordingly, disclosed herein is a robust optimization method for use in determining a desired set of parameters for an imaging system. The method can be used to optimize parameters for many different imaging devices, in particular oblique back illumination imaging devices (whether quantitative or not). Additionally, the imaging devices can be free-space, fiber-based (including endoscopic), or other handheld configurations. Though the disclosure is not so limited, below, the various embodiments of the present disclosure are discussed in the context of using a flexible fiber-optic-based qOBM system that can be applied as a handheld device or micro-endoscope for in-vivo imaging. The approaches disclosed herein can enable in silico optimization of the phase signal-to-noise-ratio (SNR) over a wide parameter space, including illumination fiber position (axial, lateral and tilt), illumination fiber numerical aperture (NA), and illumination wavelength. Sample specific scattering properties can also be taken into account.

The results provided below show that a proper combination of these parameters can provide for optimal imaging conditions, and that a single imaging device with a specific set of parameters can be optimal for multiple tissue types. Simulations were verified experimentally using tissue-mimicking phantoms. Correction was made for additional noise terms introduced by the fiber system to achieve a phase sensitivity of <20 nm with a single qOBM acquisition and a lower limit of ~ 3 nm using multiple averaged frames. The imaging capabilities of the imaging systems disclosed herein were further validated using fixed rat brain tissues from a 9 L gliosarcoma tumor model and fresh human brain tumor samples obtained directly from neurosurgery. Data show that a fiber-based qOBM system indeed recovers histological cellular information in excellent agreement with our free-space qOBM system (without stains or labels) and with hematoxylin and eosin (H&E)-stained tissue sections (after tissue processing). The methods ability to provide parameter sets for an imaging device to deliver quantitative phase contrast through a flexible fiber-based probe can be transformative for many biomedical applications, including micro-endoscopy, surgical guidance, and more. Further, the in silico optimization approach disclosed herein-which would be extremely cumbersome to perform experimentally-can be widely applied for facile optimizations of other OBM/qOBM configurations in arbitrary environments.

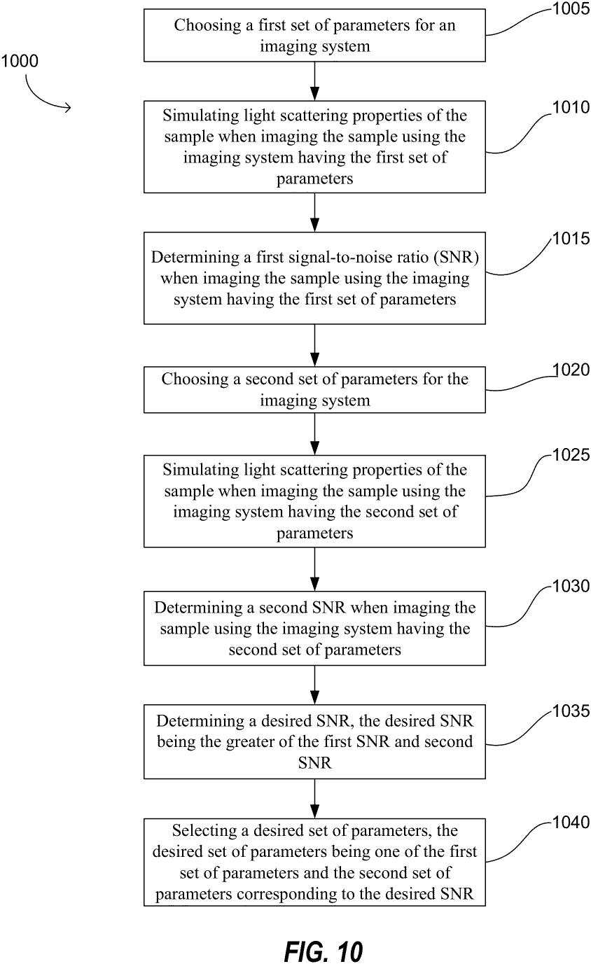

As shown in FIG. 10, an exemplary embodiment of the present disclosure provides a method of determining a desired set of parameters for an imaging system to image a sample 1000. The method can comprise choosing a first set of parameters for the imaging system 1005. Values for many different parameters that can affect the imaging capabilities of the imaging system can be chosen, which can depend on the particular imaging system being used and/or the sample-type to be imaged. For example, the parameters can include, but are not limited to, an illumination wavelength, a lateral separation distance, an axial separation distance between an MMF and GRIN lens, a MMF illuminating angle, a MMF NA, or any combination thereof.

The method can further comprise simulating light scattering properties of the sample when imaging the sample using the imaging system having the first set of parameters 1010. In some embodiments, simulating the light scattering properties of the sample 1010 can comprise simulating a measurement of the number of photons collected at a detector of the imaging device when imaging the sample using the imaging system having the first set of parameters. In some embodiments, simulating the light scattering properties of the sample 1010 can further/alternatively comprise simulating a measurement of an oblique angle of scattered photons incident a detector of the imaging sample when imaging the sample using the imaging system having the first set of parameters.

The method can further comprise determining a first signal-to-noise ratio (SNR) when imaging the sample using the imaging system having the first set of parameters 1015. Details on an exemplary method of calculating the optical transfer function are discussed below. In some embodiments, determining a first SNR 1015 can comprise calculating an optical phase transfer function for the imaging system when imaging the sample using the imaging system having the first and second sets of parameters. In some embodiments, determining a first SNR 1015 can further comprise calculating a slope of the optical transfer functions. The slope can represent an estimate of the phase sensitivity of the imaging system when imaging using the first set of parameters. In addition to or alternatively to calculating the slope of the optical transfer function, in some embodiments, determining the first SNR 1015 can include calculating an energy of the transfer function and/or calculating the maximum and/or minimum values of the optical transfer functions. In some embodiments, determining a first SNR 1015 can further comprise multiplying the slope of the corresponding optical transfer function by the square root of the number of photons collected at the detector of the imaging device when imaging the sample using the imaging system having the first set of parameters. In some embodiments, calculating a SNR 1015 can be calculating a relative SNR. In some embodiments, calculating a relative SNR can comprise multiplying the signal (e.g., product of the number of detected photons and phase sensitivity, which can be estimated a number of ways) divided by the noise (e.g., square root of the number of detected photons).

The steps of choosing a first set of parameters for the imaging system 1005, simulating light scattering properties of the sample when imaging the sample using the imaging system having the first set of parameters 1010, and determining a first signal-to-noise ratio (SNR) when imaging the sample using the imaging system having the first set of parameters 1015, can be repeated one or more times using another chosen set of parameters for the imaging system different than the first set of parameters. Thus, the method allows many combinations of the various parameters to be simulated. For example, as shown in FIG. 10, the method 1000 can further comprise: choosing a second set of parameters for the imaging system 1020; simulating light scattering properties of the sample when imaging the sample using the imaging system having the second set of parameters 1025; and determining a second SNR when imaging the sample using the imaging system having the second set of parameters 1030.

Once the SNR has been estimated for each desired set of parameters for the imaging device when imaging the sample, the method 1000 can further comprise determining a desired SNR 1035. The desired SNR can be chosen from the SNRs previously determined (e.g., the first and second SNRs, among others). For example, in some embodiments, the desired SNR can be the greater of the first SNR and second SNR. In some embodiments can be any SNR that is above a particular threshold.

The method 1000 can further comprise selecting a desired set of parameters 1040. The desired set of parameters can be the parameters corresponding to the desired SNR. For example, if the desired SNR is the first SNR, then the desired set of parameters can be first set of parameters.

In some embodiments, the method 1000 can further comprise providing an imaging device with the desired set of parameters.

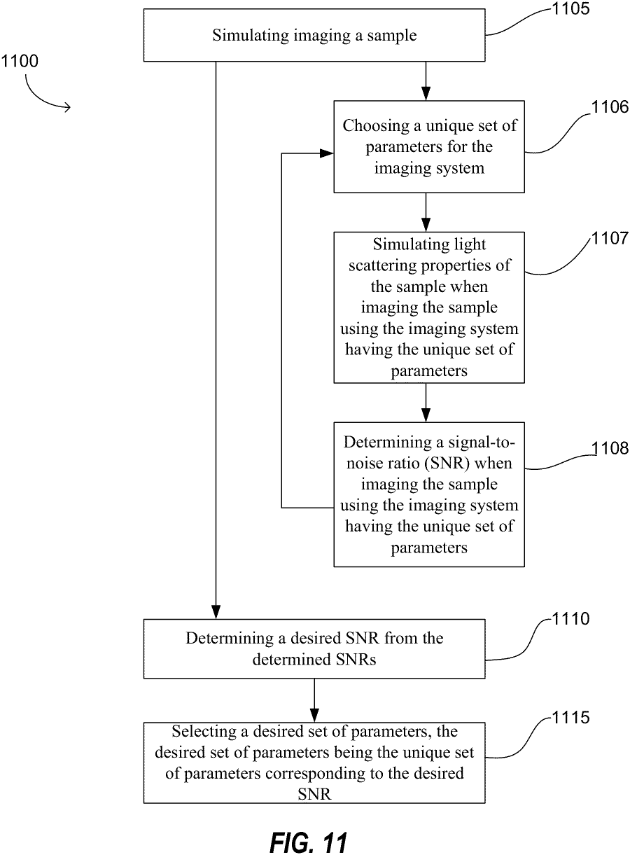

As shown in FIG. 11, another exemplary embodiment of the present disclosure provides a method of determining a desired set of parameters for an imaging system to image a sample 1100. The method 1100 can have many of the steps as method 1000. As shown in FIG. 11, the method 1100 can comprise simulating imaging a sample 1105. Simulating imaging the sample 1105 can comprise one or more iterations of the following process: choosing a unique set of parameters for the imaging system 1106; simulating light scattering properties of the sample when imaging the sample using the imaging system having the unique set of parameters 1107; and determining a signal-to-noise ratio (SNR) when imaging the sample using the imaging system having the unique set of parameters 1108. This iterative process can be iterated for each unique set of parameters to be assessed. The method 1100 can then further comprise: determining a desired SNR from the determined SNRs 1110; and selecting a desired set of parameters 1115. The desired set of parameters can be the unique set of parameters corresponding to the desired SNR

### Imaging Setup and Operation

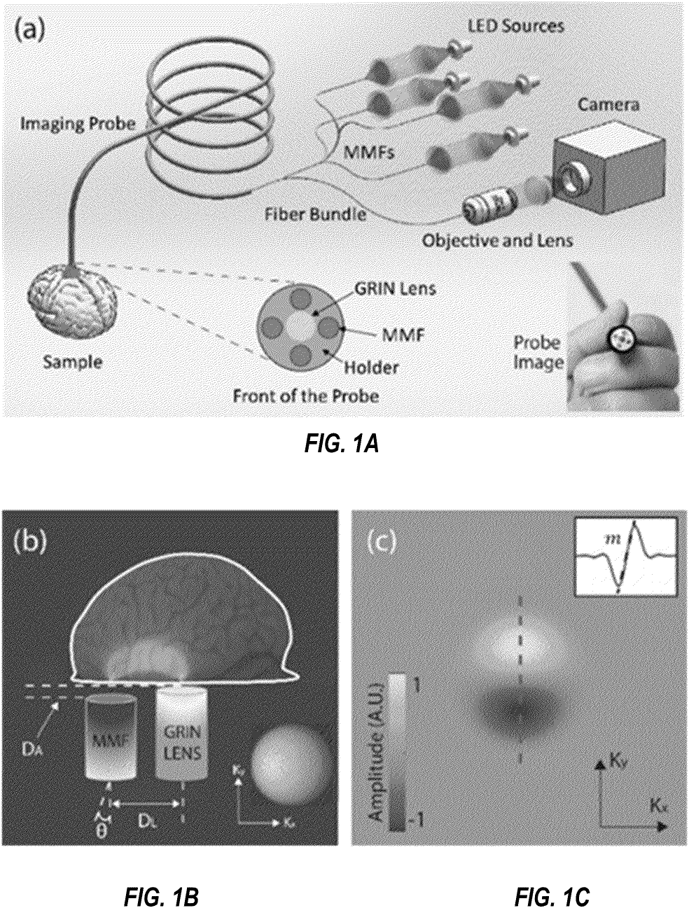

Experimental apparatus: A schematic of an experimental setup is shown in FIG. 1A. The imaging system comprises an optimized probe, a flexible fiber bundle, and a table-top camera recording setup. On the distal fiber end of the system (shown in the bottom right inset in FIG. 1A), the probe comprises a micro-GRIN objective (GRINTECH, GT-MO-080-032-ACR-VISNIR-08-00), surrounded by four MMFs for epi-illumination (Thorlabs, FP1000ERT), all held in a fabricated aluminum metal holder. The GRIN lens has a ~0.7 NA, with ~2.2X magnification and an 80 µm working distance in water. In front of the GRIN lens, a 50 µm transparent polyester film is attached to bring the focus of the GRIN lens closer to the surface of the tissue while the probe is in contact with the sample. The GRIN lens is optically glued to a flexible fiber bundle comprising 30,000 core elements (Fujikura, FIGH-30-850N). The proximal end of the fiber bundle is then connected to an imaging setup, made of a 20X objective (Olympus, RMS20X), a 150 mm achromatic doublet lens (Thorlabs, AC254-150-A), and an sCMOS camera (PCO, pco.edge 4.2 LT). For illumination, four multimode fibers (MMFs) are connected to 720 nm LEDs (Luxeon Star, SinkPAD-II) through a pair of coupling lenses (Thorlabs, ACL2520U-A). The four LEDs are individually controlled by LabView and triggered sequentially in sync with the camera acquisition. Each MMF delivers ~30 mW of power on the sample, which is well within safe limits according to the IEC 62471 safety standard. As shown in FIG. 1A, the four illuminating fibers are arranged 90 degrees apart in azimuthal angle around the GRIN lens. Captured images are then processed in real-time to obtain quantitative phase (data processing details are discussed below).

Image Processing: To retrieve quantitative phase information with qOBM, four raw intensity images can be captured, one from each LED. From the two pairs of opposed illuminations, two orthogonal differential phase contrast (DPC) images are computed via:

$${I}_{D P C} = \left({I}_{+} - {I}_{-}\right) / \left({I}_{+} + {I}_{-}\right) , \qquad \text{Equation 1:}$$

where I₊ and I_ are intensity images from opposed illuminations, and the denominator (I₊ + I_) serves as a self-normalization term. Note that the subtraction process, along with the highly incoherent illumination of multiply-scattered LED photons, enables tomographic cross-sectioning. The qualitative DPC images can be quantified with knowledge of the angular distribution of photons at the focal plane. To quantitatively model the process within a thick scattering sample, photon propagation can be numerically simulated using a Monte Carlo method. Photons are initiated at angles within the illuminating MMF NA and propagated through a stochastic scattering process given by the scattering and absorption properties of media (e.g., heterogeneous brain tissues, as in FIG. 1B). Once a large number of photons are accumulated on the simulated detector (experimentally, the focal plane of GRIN lens), the 2D optical phase transfer function can be constructed (as shown in FIG. 1C). An example angular light distribution at the focal plane is shown in the inset of FIG. 1B. The net optical transfer

$${I}_{D P C} = - \text{Im} \left\{{c}_{δ}\right\} ∗ ϕ \qquad \text{Equation 2:}$$

where c_{δ} is the point spread function, given by the Fourier transform of the 2D optical phase transfer function; c_{δ} is purely imaginary given the transfer function is odd, thus the operator Im{} takes the imaginary part; Φ is the quantitative phase; and the asterisk denotes a convolution operation. FIG. 1C shows an example optical phase transfer function, plotted in the spatial-frequency domain (kₓ, k_{y}), and its upper-right inset shows the transfer function profile along the central line. Because a single DPC image contains phase information along only one direction, a second DPC image can be acquired with orthogonal illumination relative to the first. Each DPC image can then deconvolved using a Tikhonov regularized deconvolution (regularization parameter of 3.0E-3) and averaged to obtain the qOBM quantitative phase image.

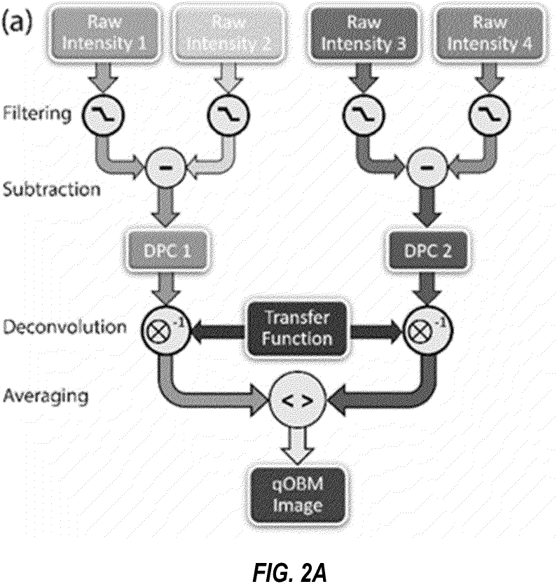

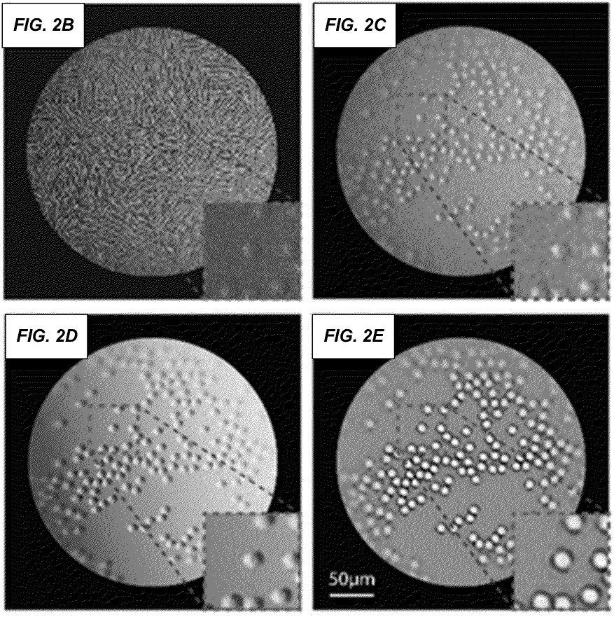

FIG. 2A illustrates the fiber-based qOBM image processing algorithm, which has an additional step from the description above. To illustrate the effects of each processing step FIGS. 2B-D present images of a phantom consisting of 10 µm polystyrene microspheres (Polysciences, Polybead 17136-5) immersed in water atop a glass slide, with a piece of paper placed underneath to serve as the scattering medium for oblique back-illumination. As illustrated in FIG. 2B, the measured raw intensity images contain a strong honeycomb pattern—a consequence of light traveling through the fiber cores but not through the cladding, which obscures the structures of interest. Numerous algorithms have been developed to suppress/remove this pattern. Here, an efficient low-pass Fourier filtering approach is used to remove the high-frequency honeycomb pattern while passing low-frequency components corresponding to the imaged scene. Specifically, a low-pass radial Butterworth (9 pole) filter is applied with a cut-off frequency of ~500 mm⁻¹, approximately corresponding to the inverse of fiber core spacing projected to the focal plane (~2 µm). An example of a filtered intensity image of the beads (one of four such images) is shown in FIG. 2C, which shows that the fiber-bundle honeycomb pattern has been effectively suppressed.

Next, FIG. 2D shows one of two DPC images obtained by subtracting two intensity images from opposed LEDs and normalizing by their sum, following Equation 1. As mentioned earlier, the subtraction and normalization processes remove out-of-focus content and absorption-related effects, thus enhancing the phase contrast. Lastly, because each DPC image contains phase gradient along only one direction, we deconvolve two orthogonal DPC images (DPC 1 and DPC 2 as in FIG. 2A) and average the retrieved results to recover the full quantitative phase. FIG. 2E shows the final quantitative phase image of the phantom. Note that the lateral spatial resolution for the fiber-based qOBM system is dictated by the fiber core spacing projected onto the focal plane (4.5 µm spacing/2.2 magnification= ~2 µm), instead of the NA of the GRIN lens (~0.7). The axial resolution is 6 µm in air, experimentally assessed using 2 µm beads. Also, note that near the edge of the field-of-view (FoV, 300 µm), there is a noticeable deterioration of image clarity due to field-curvature from the GRIN lens. This effect is noticeable in flat sample (e.g., beads on a microscope slide) but it is imperceptible in thick samples, such as biological tissues.

Imaging System Design Optimization Framework: To optimize the probe performance, several parameters can be considered for optimization, including the illumination wavelength, lateral separation distance (D_{L}) and axial separation distance (D_{A}) between MMF and the GRIN lens (see FIG. 1B), MMF illuminating angle, and MMF NA. As mentioned above, experimental optimization can be extremely cumbersome to perform over this wide parameter space, and as we show below, can easily miss optimal conditions. Thus, in some embodiments of the present disclosure, Monte Carlo simulations can be used to model the effects of the system design on the final qOBM phase image. Multiple tissue types are also modeled, as the optimized parameters can vary from tissue type to tissue type.

To analyze the impact of these parameters on the probe performance, both phase sensitivity and photon detection efficiency can be taken into account to estimate the SNR of the phase measurement. Phase sensitivity can be proportional to the central slope m of the optical phase transfer function (see inset of FIG. 1C). Alternatively, one may choose to use the total energy (area under the curve of the absolute value of the transfer function) or the maximum value. All these metrics can yield similar results and can be used as a surrogate for the phase sensitivity of a particular configuration. Thus, the signal produced by a given configuration is proportional to mN, where N is the number of photons collected at the simulated detector of the imaging system using a consistent set of initially launched photons for all numerical simulations (~1 billion). Then, the noise level, corresponding to the Poisson noise, is given by √Ñ. Therefore, the phase SNR can be estimated by SNR

$$S N R ∝ m \times \frac{N}{\sqrt{N}} = m {\sqrt{N}}_{.}$$

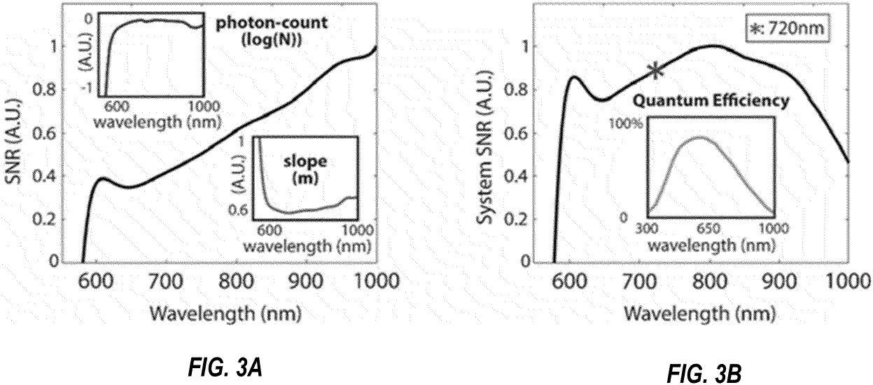

Wavelength-Dependent Optimization Factors: The illuminating wavelengths affect the imaging quality. FIG. 3A shows the calculated SNR values as a function of wavelength for brain white-matter (similar results are obtained for other tissues including grey matter and are thus not shown). For these simulations, a lateral and axial separations of D_{L} = 2.5 mm and D_{A} = 4 mm, respectively, were used between the GRIN lens and illuminating MMFs. As expected, the collected photon-count (upper-left inset of FIG. 3A) falls sharply at wavelengths below 600 nm due to hemoglobin absorptions, and remains largely unchanged over longer wavelengths. This behavior is reversed for the phase sensitivity (slope m of the transfer function). As shown in the bottom-right inset of FIG. 3A, the slope m is much larger at shorter wavelength, and falls rapidly around 600 nm. This behavior results because under large absorption (under 600 nm), photons reaching the imaging focal plane have traveled a shorter path on average (scattered fewer times), and thus possess a larger oblique angle compared to photons in less absorptive media (above 600 nm, under otherwise identical conditions). At longer wavelengths, however, the scattering coefficient decreases monotonically with respect to wavelength, resulting in a slight increase in the slope m. A lower scattering coefficient produces less diffused light, which increases the average oblique angle of photons at the focal plane, and hence increases phase sensitivity. Thus, after a local SNR peak at 600 nm, the SNR continues to increase with wavelength.

Another wavelength-dependent factor that affects the SNR is the wavelength-dependent photon detection efficiency. The most influential factor in our setup is the quantum efficiency (QE) of the camera (pco.edge 4.2 LT, inset curve in FIG. 3B). In FIG. 3B, we plot the overall system SNR, obtained by multiplying the sample-specific SNR curve in FIG. 3A by the system-specific factor √QE. Note that the detected photon-count N scales with QE, thus the SNR scales with √QE. As a result, when the wavelength increases from 600 nm to 1000 nm, the falling QE lowers the overall system SNR, and leaves a relatively high-SNR region from ~600 nm-950 nm with a maximum around 800 nm. The operating wavelength used is at 720 nm, which resides well within the high-SNR region.

Geometrical Optimization Factors: Probe/imaging device geometrical factors also affect SNR, including lateral and axial separation distances (D_{L} and D_{A}, respectively, as in FIG. 1B) between the illuminating MMF and the GRIN lens, as well as the MMF illumination angle and NA. Simulations were performed using optical properties of brain tissues (white and grey matter), breast tissue, and epidermis. Due to physical size constraints of the probe (GRIN lens metal housing of 1.4 mm in diameter and MMF bare fiber of 1 mm in diameter) and the fact that GRIN lens works in contact mode, conditions were simulated with D_{L} ∈ [1.5 mm -5 mm] and D_{A} ∈ [0 mm - 6 mm]. Fiber illumination angles of 0 to 90 degrees and NAs of 0 to 1 were considered.

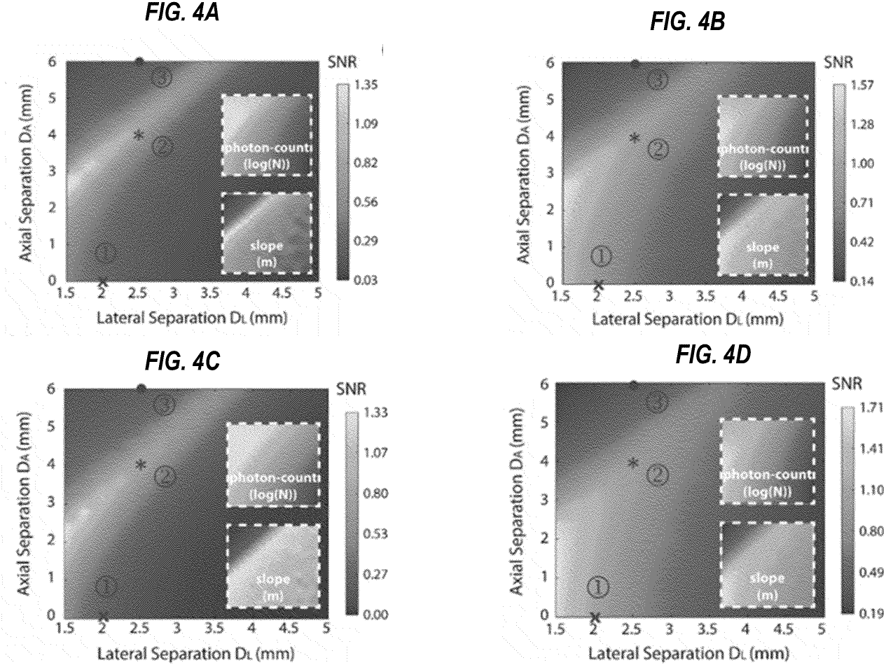

We begin with an analysis of SNR as a function of D_{L} and D_{A} using a constant 0.5 NA fiber without tilting the illumination MMF angle. SNR results are plotted in FIG. 4 for four different tissue types. Points denoted by ①, ②, and ③ indicate geometries that are experimentally validated below using scattering phantoms that mimic brain white and grey matter. Insets plot the detected photon-count N (in logarithm scale) and central slope m of the optical transfer function.

As shown in the insets of FIG. 4, detected photon-counts N are higher in the low-D_{L}/high-D_{A} regions. This is because (1) with a lower D_{L}, the MMF (light source) and GRIN lens (detector) are closer to each other and more photons are thus detected, and (2) with a higher D_{A}, more photons from the MMF will be illuminating regions closer to the detected focal plane area. Meanwhile, the phase sensitivity assessed via slope m (FIG. 4 insets) shows a significantly different behavior with a set of maxima near the edges of the high photon-count regions and with decreasing values elsewhere. This behavior can be explained as follows: when the MMF light source is far from the GRIN lens (low photon-count region), detected photons will have a more randomized angular distribution, thus lowering the average angle of detected photons and the slope m, which indicates a lower phase sensitivity. On the other hand, when the source is too close to the detector (high photon-count region), photons will not experience enough scattering to significantly alter their original forward propagation direction. Therefore, there is an optimal region of operation where (1) sufficient photons are collected and (2) incident light has had sufficient opportunity to scatter such that photons change trajectory and illuminate the focal plane obliquely.

All four tissue types simulated here (white matter, grey matter, epidermis, breast tissue) form an optimal peninsula-shaped high-SNR region with slightly different shapes and optimal regions. However, optimal regions for these tissues do show substantial overlap. This indicates that a single probe/imaging device geometry can be produced with fairly optimal conditions for a wide variety of biological specimens. However, given that optimal regions can be narrow and not necessarily in intuitive configurations, this type of analysis may be warranted when optimizing probes for use in tissues with different optical properties.

The SNR dependence with MMF illumination angle and NA can now be considered, with varying separation distances D_{A} and D_{L}. Note that, in practice, manipulating the fiber angle in the probe design may be more difficult than changing the axial and lateral separation distances. Keeping the fiber angle at zero degrees (parallel to GRIN lens) provides the simplest geometrical configuration, with the least chance of breaking a fiber. If fiber polishing is used to alter the initial illumination angle, the available angular range can be quite limited (~0 to 15 degrees) due to total internal reflections. Similarly, there are few options to fine tune the fiber NA, with 0.1, 0.3 and 0.5 NAs being the most common commercially available options. Nevertheless, it is still instructive to show how these factors influence the SNR. Given the large parameter space, only simulated imaging conditions in grey matter are shown, but the same approach can be readily implemented for other tissue types (indeed white matter, epidermis and breast tissues show similar behavior).

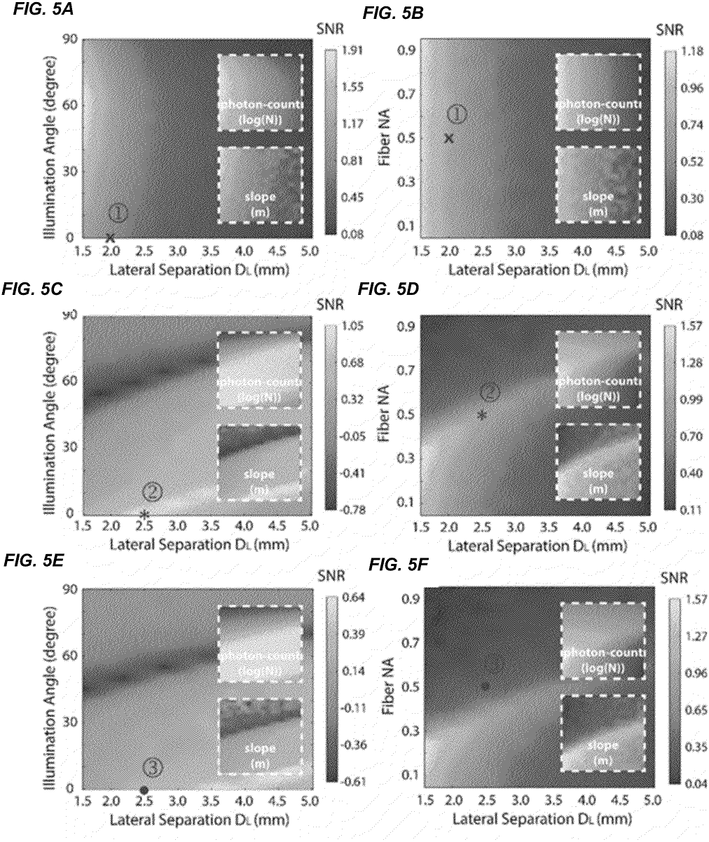

The first row of FIG. 5 (with D_{A}=0) shows that SNR has little to no dependence with the fiber illumination angle or NA. However, as D_{A}>0, both illumination angle and fiber NA have a significant influence over the SNR. The second and third rows of FIG. 5 show two examples with D_{A} = 4 mm and 6 mm, respectively. The SNR, as a function of fiber NA and lateral separation (second column in FIG. 5), show peninsula-shaped high-value regions, similar to the axial and lateral separation plot in FIG. 4. This indicates that changing NA has a similar effect on SNR as changing the axial separation. The SNR dependence with fiber illumination angle, however, is more complex (first column in FIG. 5). FIGS. 5C & 5E show that as the angle increases, the SNR goes from positive to negative, reaching zero at around ~30-60 degrees (teal color region). After reaching its most negative value (dark blue region) the SNR approaches zero again as the angle increases further. This behavior is a result of two factors. First, from 0-30 degrees the slope m drops from its highest value into a relative uniform-value region, due to the loss of overall illumination obliquity. After the illumination passes ~30-60 degrees, however, the net angular distribution of the illuminating light at the imaging focal plane appears to come from the opposite direction, resulting in a negative slope m of the transfer function (hence the negative SNR). After reaching a minimum slope (which corresponds to high sensitivity but with an opposite shear direction in the DPC image), the slope begins to approach zero again (low sensitivity) resulting from a loss of light obliquity of light at the focal plane due to more scattering. The second factor affecting SNR is the detected photon count (shown in FIGS. 5C & 5E insets as log(N)). At illumination angles below ~60 degrees, detected photon counts can be relatively high, but beyond this region counts can decrease sharply due to the overly large tilting angle preventing adequate collection of photons within the acceptance angels (NA) of the GRIN lens.

These results demonstrate that designing the probe and finding its optimal operation can be done by understanding of a wide parameter space and the physics behind the photon ensemble distribution inside thick samples. Experimentally performing this optimization can be cumbersome and could lead to less than optimal designs. Geometry^{②} shows fairly optimal performance over the large parameter space considered here.

Experimental Validations Using Tissue Mimicking Scattering Phantoms: Simulations were verified experimentally using scattering phantoms that mimic brain white and grey matter. The scattering phantoms used polydimethylsiloxane (PDMS) (Dow, Sylgard 184, with a 10:1 curing agent ratio) as the substrate, titanium dioxide (TiO₂, Atlantic Equipment Engineers, Ti-602) as the scattering agent, and India ink (Pro Art, PRO-4100) as the absorbing agent. The concentration of TiO₂ was set to 3.94 g/L for white matter and 1.57 g/L for grey matter, while the concentration of the India ink was 0.218 g/L for both (the absorption coefficients of white and grey matter are approximately equal at 720 nm). On top of these scattering phantoms, 10 µm polystyrene beads were placed in water which serve as the phase target to image.

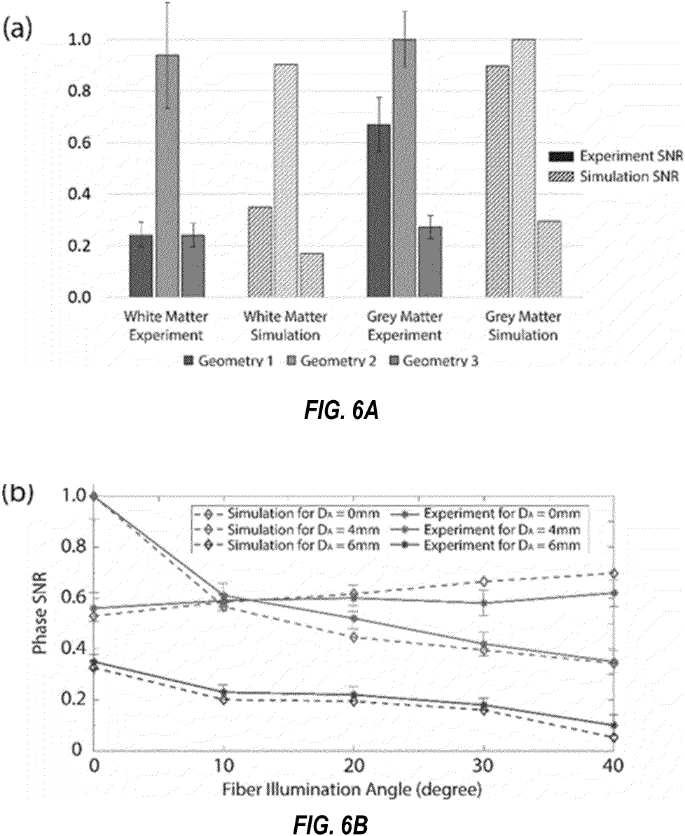

Three probes were fabricated with different lateral and axial separations (D_{L}, D_{A}), namely, geometry^{①}: (2 mm, 0 mm), geometry^{①}: (2.5 mm, 4 mm), and geometry^{①}: (2.5 mm, 6 mm) as marked in red circles in FIGS. 4-5. These three geometries were specifically chosen to test the validity of the model, which has an unusual peninsula-shaped high-SNR region, and has a slight level of tissue dependence (FIGS. 4A-D). Note that geometry^{③} is out of the peninsula-shaped high-SNR region and geometry^{②} is well within the optimal SNR region for white and grey matter ((FIGS. 4A-B, respectively)), while geometry^{①} is outside the optimal region only for white matter but not for grey matter. For each probe geometry and phantom type, 10 beads and 10 featureless areas were measured in the FoV, and then the experiment SNR was computed using the average phase value of the beads, divided by the average phase-standard-deviation of the featureless areas. FIG. 6A compares experimental SNRs to simulation. For ease of comparison, all SNR values are normalized to geometry^{②} value in the grey matter phantom.

As FIG. 6A shows, the experiment and simulation SNR values were in good agreement for all configurations and tissue types. Geometry^{②}exhibited the highest SNR and geometry^{③} had the lowest SNR among all cases. As expected, geometry ^{①} yielded a tissue-type dependent SNR, with the configuration providing a better SNR for grey matter compared to white matter. These experimental results validate our numerical treatment, including the peninsula-shaped high-SNR region and the slight tissue dependence. These results also show the importance of careful probe optimization to achieve optimal operation conditions (for brain imaging in this case). For instance, the most intuitive geometry of D_{A}= 0 with a small D_{L}, as with geometry^{①}, leads to vastly sub-optimal performance in white matter, which can be remedied with a slightly less obvious geometry, using small offset in D_{A} as with geometry^{②}.

The SNR as a function of fiber illumination angle at multiple axial separation distances was experimentally measured (FIG. 6B). The fiber angle was varied from 0 degree to 40 degrees, with respect to the GRIN-lens. Similar to FIG. 5, three different axial separations (D_{A} =0 mm, 4 mm, 6 mm) were considered with a fixed lateral distance D_{L} = 2.5 mm. Once again, the 10 µm polystyrene beads immersed in water atop a grey matter were measured mimicking scattering phantom. For each experimental SNR, the phase value obtained from 10 beads was measured and divided by the phase standard deviations from featureless background areas to estimate the SNR. As shown in FIG. 6B, the measured experimental SNRs were in excellent agreement with the simulation results. Importantly, geometry^{②} (curve intersecting the Phase SNR axis at ~1.0 in FIG. 6B with zero-degree fiber illumination angle) shows an optimal SNR configuration. This is also in agreement with the unique optimal behavior predicted by the model in FIGS. 5A, 5C, & 5E, in which an axial separation distance of 4 mm showed an optimal SNR at zero-degree illumination angle, but then falls-off sharply (i.e., SNR worsens) as the illumination angle is increased. In comparison, the other axial separation distances (0 mm and 6 mm) with fixed D_{L} = 2.5 mm show a weaker dependance with illumination angle. Again, this unique behavior predicted by the model is in excellent agreement with the experiments.

Below, geometry^{②} was adopted because this configuration achieves optimal conditions (i.e., highest SNR) for white and grey matter (as well as other tissue types) as shown in FIGS. 4-6.

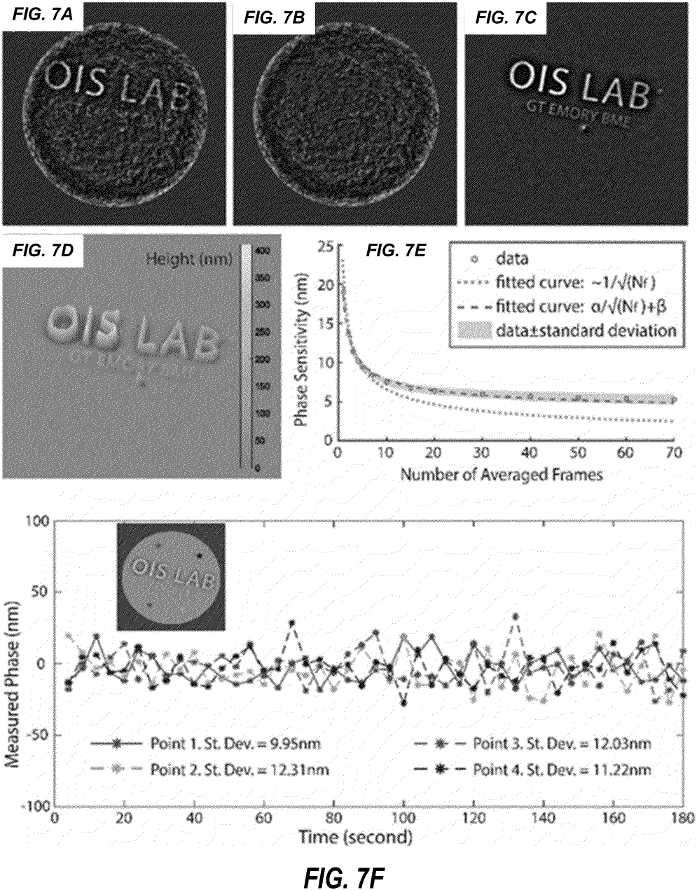

Characterization of Imaging System Phase Sensitivity: The phase sensitivity of the imaging system can be quantified using a photo-lithographic quartz target consisting of letters of different heights (“OIS”: 300 nm, “LAB”: 200 nm, and “GT EMORY BME”: 100 nm). In this case, a 1% intralipid agar phantom was used as the scattering medium below the phase target (this mimics previous experimental conditions to assess sensitivity and permits direct comparison to the free-space qOBM system). As shown in FIG. 7A, the phase target contains letter structures that are comparable in phase-values to the featureless background. To characterize the spatial fluctuations and obtain a quantitative estimate of phase sensitivity, four rectangular featureless areas (40 µm-by-40 µm) were selected across the FoV and their average phase standard deviations were characterized. This resulted in a phase sensitivity of ~0.58 rad which translated to a ~67 nm sensitivity for this sample. However, while the background phase structures appear to be random, the pattern is mostly static and is a result of phase irregularities among fiber cores in the fiber bundle. FIG. 7B shows an image of a blank area which has a high degree of similarity to the background in FIG. 7A. It was also observed that the pattern did not fluctuate significantly as the fiber bundle was moved. Thus, to eliminate the static phase noise, a qOBM image of a blank region (FIG. 7(b)) was taken and subtracted from subsequent acquisitions. FIGS. 7C-D illustrate the drastic improvements achieved by the simple background subtraction. Using the same regions as before, a phase sensitivity of ~0.17 rad or ~19 nm was obtained for this sample, which is over a three-fold improvement.

Lastly, it was explored how averaging multiple frames helps to mitigate noise and improve the phase sensitivity. The phase sensitivity in FIG. 7E was calculated using the same four rectangular featureless areas (40 um-by-40 um) as before. As FIG. 7E illustrates, the phase sensitivity (in nm) of the probe first decreases (i.e., improves) quickly with increasing number of averaged frames (N_{f}), roughly following a

$$\sim 1 / \sqrt{{N}_{f}}$$

Poisson-noise distribution (dotted green line). However, after averaging more than ~10 frames, the sensitivity improvement slows down and begins to deviate from the expected shot noise behavior. This deviation was designated to persistent noise from the fiber bundle that is not completely eliminated by the background subtraction correction. By fitting the data to a slightly different model following

$$α / \sqrt{{N}_{f}} + β ,$$

with α being a proportionally constant and β a lower sensitivity limit set by the fiber bundle, a much better fit (dashed line within the shaded line) was obtained. It was found that β = 3.05 nm, which effectively represents the best-case-scenario sensitivity (when large averaging can be tolerated). It is likely that different fiber-bundles could show different lower sensitivity limit coefficients (β). By averaging 70 frames, the probe sensitivity was ~0.05 rad or ~5.4 nm, which was comparable to our previous free-space qOBM system’s sensitivity.

Finally, it was considered how the power stability of the light source (LEDs) can lead to noise in the measured phase value. To estimate the effect, a power meter (Thorlabs, PM100D) was used to measure the delivered LED power from one single illumination fiber. Its standard deviation was ~0.030 mW or ~0.1% power fluctuations over a period of 3 minutes. The impact of the power instability on the measured phase can be analyzed by investigating at measured phase values from single-pixels over time. In FIG. 7F, our measurements are included on four independent pixels, and their measured phase values over a period of three minutes are analyzed. The temporal phase standard deviations as measured by these four representative background points was ~12 nm. Recall that the spatial phase variations (assessed from 40 µm-by-40 µm featureless areas) are ~19 nm, which indicates that the temporal phase noise is a slightly less severe noise factor than the spatial phase noise (i.e., fixed pattern noise from fiber bundle, remaining even after conducting the background subtraction).

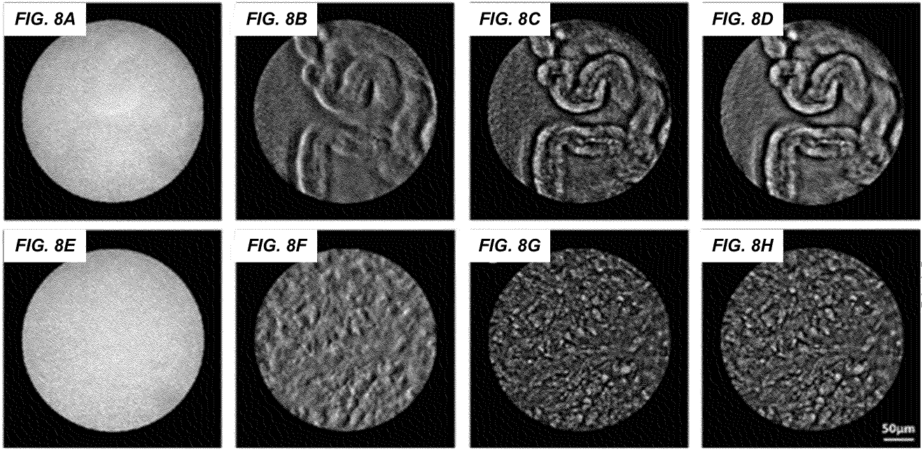

Imaging System Validation Using Fixed Rat Brain and Freshly Excised Human Brain Tumor Samples: To demonstrate the imaging capability of the fiber-based qOBM system on biological samples, formalin-fixed, excised rat brain samples from a 9 L gliosarcoma rat tumor model were measured. FIG. 8 shows the measurements of a cortex structure (choroid plexus) in a healthy rat brain (FIGS. 8A-D) and a dense tumor region of the 9L tumor model (FIGS. 8E-H). Specifically, FIGS. 8A & 8E show the low-pass filtered, single-frame intensity images (from one LED), where little or no identifiable detail of the tissue can be seen. In FIGS. 8B & 8F, a DPC image (with no frame averaging) is shown where some tissue structure starts to appear with horizontal phase gradient information. FIGS. 8C & 8G show the retrieved quantitative phase (qOBM image) with background subtraction (no frame averaging). Here, tissue structures are more conspicuous with appreciable detail of the folded structures of choroid plexus (FIG. 8C) and the characteristic granular cellular structures of the 9L tumor model (FIG. 8G). FIGS. 8D & 8H show the same structures after averaging over 40 qOBM images, which has a higher SNR, but this may only be possible for still samples.

As a final demonstration of the capabilities of the fiber-based probe, qOBM images of freshly excised human brain tumor samples (astrocytomas) discarded from neurosurgery were acquired. For comparison, qOBM images using our free-space qOBM system were also acquired. After imaging with qOBM, samples were furthered processed for histology to obtain the “gold-standard” H&E-stained bright-field images for comparison.

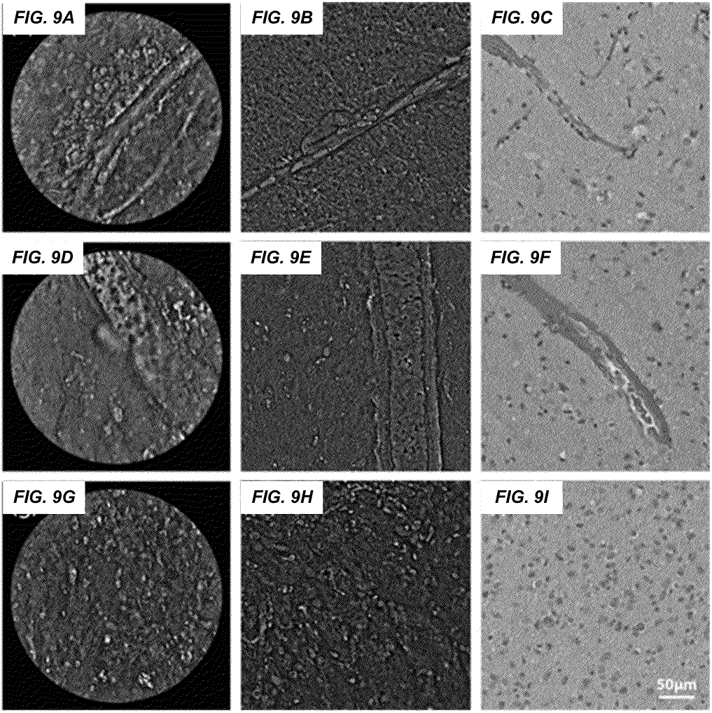

Measurements are shown in FIG. 9. The first column shows the fiber-based qOBM images (no averaging, single qOBM acquisition at 10 Hz), and the second and third columns show images from our free-space qOBM system and H&E, respectively. Images in each row are from the same specimen (and hence patient and tissue type). Note that, while the three types of images (probe-qOBM, free-space-qOBM, and H&E) capture similar structures from the same specimen, they are not necessarily from the exact same spot. FIGS. 9A-C present an area with a capillary blood vessel (single blood cells inside) and some tumor cells around/along it. FIGS. 9D-F show a larger blood vessel with many blood cells inside and some astrocytoma (tumor) cells nearby. FIGS. 9G-I show a densely packed tumor cell area, with highly myelinated processes present, which are more prominent in qOBM than H&E. Our fiber-based qOBM system can measure clear histological structures from unstained, thick, fresh samples comparable to the free-space qOBM and H&E, which illustrates the potential use of our flexible probe for in-vivo, intraoperative diagnosis of human brain tumors, among many other applications.

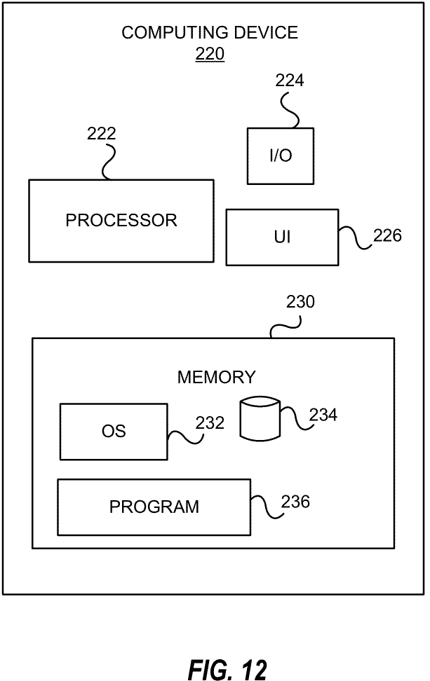

FIG. 12 illustrates an examplary computing device configured the methods (or one or more steps of the methods) disclosed herein. As will be appreciated by one of skill in the art, the computing device 220 can be configured to implement all or some of the features described in relation to the methods 1000 1100. As shown, the computing device 220 may include a processor 222, an input/output (“I/O”) device 224, a memory 230 containing an operating system (“OS”) 232 and a program 236. In certain example implementations, the computing device 220 may be a single server or may be configured as a distributed computer system including multiple servers or computers that interoperate to perform one or more of the processes and functionalities associated with the disclosed embodiments. In some embodiments, computing device 220 may be one or more servers from a serverless or scaling server system. In some embodiments, the computing device 220 may further include a peripheral interface, a transceiver, a mobile network interface in communication with the processor 222, a bus configured to facilitate communication between the various components of the computing device 220, and a power source configured to power one or more components of the computing device 220.

A peripheral interface, for example, may include the hardware, firmware and/or software that enable(s) communication with various peripheral devices, such as media drives (e.g., magnetic disk, solid state, or optical disk drives), other processing devices, or any other input source used in connection with the disclosed technology. In some embodiments, a peripheral interface may include a serial port, a parallel port, a general-purpose input and output (GPIO) port, a game port, a universal serial bus (USB), a micro-USB port, a high definition multimedia interface (HDMI) port, a video port, an audio port, a Bluetooth ™ port, a near-field communication (NFC) port, another like communication interface, or any combination thereof.

In some embodiments, a transceiver may be configured to communicate with compatible devices and ID tags when they are within a predetermined range. A transceiver may be compatible with one or more of: radio-frequency identification (RFID), near-field communication (NFC), Bluetooth ™, low-energy Bluetooth ™ (BLE), WiFi™, ZigBee™, ambient backscatter communications (ABC) protocols or similar technologies.

A mobile network interface may provide access to a cellular network, the Internet, or another wide-area or local area network. In some embodiments, a mobile network interface may include hardware, firmware, and/or software that allow(s) the processor(s) 222 to communicate with other devices via wired or wireless networks, whether local or wide area, private or public, as known in the art. A power source may be configured to provide an appropriate alternating current (AC) or direct current (DC) to power components.

The processor 222 may include one or more of a microprocessor, microcontroller, digital signal processor, co-processor or the like or combinations thereof capable of executing stored instructions and operating upon stored data. The memory 230 may include, in some implementations, one or more suitable types of memory (e.g. such as volatile or non-volatile memory, random access memory (RAM), read only memory (ROM), programmable read-only memory (PROM), erasable programmable read-only memory (EPROM), electrically erasable programmable read-only memory (EEPROM), magnetic disks, optical disks, floppy disks, hard disks, removable cartridges, flash memory, a redundant array of independent disks (RAID), and the like), for storing files including an operating system, application programs (including, for example, a web browser application, a widget or gadget engine, and or other applications, as necessary), executable instructions and data. In one embodiment, the processing techniques described herein may be implemented as a combination of executable instructions and data stored within the memory 230.

The processor 222 may be one or more known processing devices, such as, but not limited to, a microprocessor from the Pentium™ family manufactured by Intel™ or the Turion ™ family manufactured by AMD™. The processor 222 may constitute a single core or multiple core processor that executes parallel processes simultaneously. For example, the processor 222 may be a single core processor that is configured with virtual processing technologies. In certain embodiments, the processor 222 may use logical processors to simultaneously execute and control multiple processes. The processor 222 may implement virtual machine technologies, or other similar known technologies to provide the ability to execute, control, run, manipulate, store, etc. multiple software processes, applications, programs, etc. The processor 222 may also comprise multiple processors, each of which is configured to implement one or more features/steps of the disclosed technology. One of ordinary skill in the art would understand that other types of processor arrangements could be implemented that provide for the capabilities disclosed herein.

In accordance with certain example implementations of the disclosed technology, the computing device 220 may include one or more storage devices configured to store information used by the processor 222 (or other components) to perform certain functions related to the disclosed embodiments. In one example, the computing device 220 may include the memory 230 that includes instructions to enable the processor 222 to execute one or more applications, such as server applications, network communication processes, and any other type of application or software known to be available on computer systems. Alternatively, the instructions, application programs, etc. may be stored in an external storage or available from a memory over a network. The one or more storage devices may be a volatile or non-volatile, magnetic, semiconductor, tape, optical, removable, non-removable, or other type of storage device or tangible computer-readable medium.

In one embodiment, the computing device 220 may include a memory 230 that includes instructions that, when executed by the processor 222, perform one or more processes consistent with the functionalities disclosed herein. Methods, systems, and articles of manufacture consistent with disclosed embodiments are not limited to separate programs or computers configured to perform dedicated tasks. For example, the computing device 220 may include the memory 230 that may include one or more programs 236 to perform one or more functions of the disclosed embodiments.

The processor 222 may execute one or more programs located remotely from the computing device 220. For example, the computing device 220 may access one or more remote programs that, when executed, perform functions related to disclosed embodiments.

The memory 230 may include one or more memory devices that store data and instructions used to perform one or more features of the disclosed embodiments. The memory 230 may also include any combination of one or more databases controlled by memory controller devices (e.g., server(s), etc.) or software, such as document management systems, Microsoft™ SQL databases, SharePoint™ databases, Oracle™ databases, Sybase™ databases, or other relational or non-relational databases. The memory 230 may include software components that, when executed by the processor 222, perform one or more processes consistent with the disclosed embodiments. In some examples, the memory 230 may include a database 234 configured to store various data described herein. For example, the database 234 can be configured to store the software repository 102 or data generated by the repository intent model 104 such as synopses of the computer instructions stored in the software repository 102, inputs received from a user (e.g., responses to questions or edits made to synopses), or other data that can be used to train the repository intent model 104.

The computing device 220 may also be communicatively connected to one or more memory devices (e.g., databases) locally or through a network. The remote memory devices may be configured to store information and may be accessed and/or managed by the computing device 220. By way of example, the remote memory devices may be document management systems, Microsoft™ SQL database, SharePoint™ databases, Oracl™ databases, Sybase™ databases, or other relational or non-relational databases. Systems and methods consistent with disclosed embodiments, however, are not limited to separate databases or even to the use of a database.

The computing device 220 may also include one or more I/O devices 224 that may comprise one or more user interfaces 226 for receiving signals or input from devices and providing signals or output to one or more devices that allow data to be received and/or transmitted by the computing device 220. For example, the computing device 220 may include interface components, which may provide interfaces to one or more input devices, such as one or more keyboards, mouse devices, touch screens, track pads, trackballs, scroll wheels, digital cameras, microphones, sensors, and the like, that enable the computing device 220 to receive data from a user.

In example embodiments of the disclosed technology, the computing device 220 may include any number of hardware and/or software applications that are executed to facilitate any of the operations. The one or more I/O interfaces may be utilized to receive or collect data and/or user instructions from a wide variety of input devices. Received data may be processed by one or more computer processors as desired in various implementations of the disclosed technology and/or stored in one or more memory devices.

While the computing device 220 has been described as one form for implementing the techniques described herein, other, functionally equivalent, techniques may be employed. For example, some or all of the functionality implemented via executable instructions may also be implemented using firmware and/or hardware devices such as application specific integrated circuits (ASICs), programmable logic arrays, state machines, etc. Furthermore, other implementations of the computing device 220 may include a greater or lesser number of components than those illustrated

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the U.S. Pat. and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

What is claimed is:

1. A method of determining a desired set of parameters for an imaging system to image a sample, the method comprising:
choosing a first set of parameters for the imaging system;
simulating light scattering properties of the sample when imaging the sample using the imaging system having the first set of parameters;
determining a first signal-to-noise ratio (SNR) when imaging the sample using the imaging system having the first set of parameters;
choosing a second set of parameters for the imaging system;
simulating light scattering properties of the sample when imaging the sample using the imaging system having the second set of parameters;
determining a second SNR when imaging the sample using the imaging system having the second set of parameters;
determining a desired SNR, the desired SNR being the greater of the first SNR and second SNR; and
selecting a desired set of parameters, the desired set of parameters being one of the first set of parameters and the second set of parameters corresponding to the desired SNR.

2. The method of claim 1, wherein simulating light scattering properties of the sample when imaging the sample using the imaging system having the first set of parameters and simulating light scattering properties of the sample when imaging the sample using the imaging system having the second set of parameters each comprises simulating a measurement of the number of photons collected at a detector of the imaging device when imaging the sample using the imaging system having the first and second sets of parameters, respectively.

3. The method of claim 2, wherein simulating light scattering properties of the sample when imaging the sample using the imaging system having the first set of parameters and simulating light scattering properties of the sample when imaging the sample using the imaging system having the second set of parameters each comprises simulating a measurement of an oblique angle of scattered photons incident a detector of the imaging sample when imaging the sample using the imaging system having the first and second sets of parameters, respectively.

4. The method of claim 2, wherein determining a first SNR and determining a second SNR each comprises calculating an optical phase transfer function for the imaging system when imaging the sample using the imaging system having the first and second sets of parameters, respectively.

5. The method of claim 4, wherein determining a first SNR and determining a second SNR each further comprises calculating a slope of the optical transfer functions.

6. The method of claim 5, wherein determining a first SNR and determining a second SNR each further comprises multiplying the slope of the corresponding optical transfer function by the square root of the number of photons collected at the detector of the imaging device when imaging the sample using the imaging system having the first and second sets of parameters, respectively.

7. The method of claim 1, wherein each of the first set of parameters and second set of parameters comprises an illumination wavelength.

8. The method of claim 1, wherein each of the first set of parameters and second set of parameters comprises a lateral separation distance.

9. The method of claim 1, wherein each of the first set of parameters and second set of parameters comprises an axial separation distance between an MMF and GRIN lens.

10. The method of claim 1, wherein each of the first set of parameters and second set of parameters comprises a MMF illuminating angle.

11. The method of claim 1, wherein each of the first set of parameters and second set of parameters comprises a MMF NA.

12. The method of claim 1, wherein the imaging system is a quantitative oblique back-illumination microscopy (qOBM) system.

13. The method of claim 1, wherein the imaging system is an endoscopic oblique back illumination imaging system.

14. A method of determining a desired set of parameters for an imaging system to image a sample, the method comprising:
simulating imaging a sample, comprising one or more iterations of:
choosing a unique set of parameters for the imaging system;
simulating light scattering properties of the sample when imaging the sample using the imaging system having the unique set of parameters; and
determining a signal-to-noise ratio (SNR) when imaging the sample using the imaging system having the unique set of parameters;
determining a desired SNR from the determined SNRs; and
selecting a desired set of parameters, the desired set of parameters being the unique set of parameters corresponding to the desired SNR.

15. The method of claim 14, wherein simulating light scattering properties of the sample when imaging the sample using the imaging system having the unique set of parameters comprises simulating a measurement of the number of photons collected at a detector of the imaging device when imaging the sample using the imaging system having the unique set of parameters.

16. The method of claim 15, wherein simulating light scattering properties of the sample when imaging the sample using the imaging system having the unique set of parameters comprises simulating a measurement of an oblique angle of scattered photons incident a detector of the imaging sample when imaging the sample using the imaging system having the unique set of parameters.

17. The method of claim 15, wherein determining the SNR comprises calculating an optical phase transfer function for the imaging system when imaging the sample using the imaging system having the unique set of parameters.

18. The method of claim 17, wherein determining the SNR further comprises calculating a slope of the optical transfer function.

19. The method of claim 18, wherein determining the SNR further comprises multiplying the slope of the optical transfer function by the square root of the number of photons collected at the detector of the imaging device when imaging the sample using the imaging system having the unique set of parameters.

20. The method of claim 14, wherein the unique set of parameters comprises one or more selected from the following: an illumination wavelength; a lateral separation distance; an axial separation distance between an MMF and GRIN lens; a MMF illuminating angle; and a MMF NA.
